(19) United States
(12) Patent Application Publication
(10) Pub. No.: US 2005/0176674 A1
(43) Pub. Date: Aug. 11, 2005

(54) Composition and method for use in cartilage affecting conditions

(76) Inventors: Kim Gene Friesen, Topeka, KS (US); Philip W. Toll, Valley Falls, KS (US)

Correspondence Address: Colgate-Palmolive Company, 909 River Road, P.O. Box 1343, Piscataway, NJ, 08855-1343, US

(21) Appl. No.: 10/774,781

(22) Filed: Feb. 9, 2004

(51) Int. Cl. A61K 31/7076; A61K 31/198; A61K 31/28

(52) U.S. Cl. 514/46; 514/184; 514/562; 514/492

(57) ABSTRACT

A method for decreasing cartilage abnormalities in an animal in need of such decrease which comprises systemically administering to said animal a cartilage abnormality decreasing effective amount of a combination of at least one sulfur containing amino acid and manganese.

### BACKGROUND OF THE INVENTION

Virtually all joints have cartilage. Cartilage is important in the body of animals for providing flexibility, compressability under pressure, cushion, tensile strength, ranges and smoothness of movement within joints. Healthy, well developed cartilage is relatively resistant to deterioration over time. Poorly developed cartilage is more susceptible to damage that leads to disease. Examples of joints having cartilage include fingers and toes, neck, knee, hip, shoulder and the like. Animals can suffer from a number of conditions where cartilage is negatively affected thereby bringing about a reduction in the joint's flexibility, compressability and often times resulting in a generalized inflammation of the joint and/or tissue surrounding the joints. Such animal then has significant loss of joint function and experiences pain. Another measure of cartilage health is the quantity of abnormalities visually on the cartilage observed after sacrifice of the animal. The higher the abnormalities, the further the overall joint is weakened which makes it more susceptible to a condition or exacerbates an existing condition. These conditions include arthritis, osteo and rheumatoid, osteochondrosis, degenerative joint disease, synovitis, bacterial purulent arthritis, osteoarthropathia and psoriatica among others. The visualized cartilage abnormalities include lesions in general, erosions, and abnormal growths. Other ways of observing cartilage abnormalities without sacrifice of the animal include MRI, computerized tomography and radiography.

We have now found a method and composition for reducing the quantity of cartilage abnormalities in those animals in need of said assistance. These animals can already have classical symptoms of the condition(s) or can be susceptible of such condition(s), the latter for example being a large breed dog having hip dysplasia problems which can bring about arthritis or similar conditions. Such assistance can even be given to animals in no apparent immediate need of such assistance but wherein growth of cartilage occurs as in the younger years or approaching an age where such conditions are relatively commonplace, for example “old age”.

The assistance is provided by the use of certain quantities of a sulfur containing amino acid, such as methionine, and manganese administered in a systemic manner, such as orally, in a food, liquid or dosage unit form. The data in the specification shows that cartilage abnormalities as measured visually are substantially decreased using the invention. The reduction of cartilage abnormalities enhance the joint health and make the joint less susceptible to physical damage and cartilage destruction conditions such as arthritis and other conditions which attack the joint and cartilage.

### SUMMARY OF THE INVENTION

In accordance with the invention, there is a method for decreasing cartilage abnormalities in an animal in need of such decrease which comprises administering to said animal a cartilage abnormality decreasing effective amount of at least one sulfur containing amino acid and manganese.

Another aspect of the invention is a method for preventing degradation of cartilage tissue in an animal in need of said prevention which comprises administering to said animal a cartilage degradation prevention effective amount of at least one sulfur containing amino acid and manganese.

An additional aspect of the invention is a composition suitable for systemic administration to an animal comprising a cartilage abnormality decreasing amount of at least one sulfur containing amino acid and manganese in association with a carrier.

Further, the various modes of action of the sulfur containing amino acid and manganese can bring about each or a combination of the following effects:
(a) enhancing cartilage development in an animal;
(b) preventing disease associated with cartilage degradation in an animal; and
(c) treating disease associated with cartilage degradation in an animal. All of such above effects can be directed to animals in need thereof.

There are many other aspects of the invention disclosed throughout this specification.

### DETAILED DESCRIPTION OF THE INVENTION

An animal as used throughout the specification includes human, dog, cat, horse, goat, sheep, swine, cattle, birds including turkeys and chickens, and the like. Preferred are humans, dogs, cats, horses and swine.

Cartilage affecting conditions wherein cartilage abnormalities are significant are those which are particularly managed by the administration of the sulfur containing amino acid and manganese. Illustrative examples of such conditions include osteoarthritis, rheumatoid arthritis, osteochondrosis, degenerative joint disease, synovitis, bacterial purulent arthritis, osteoarthropathia, and psoriatica.

The active material(s) of the invention can be administered in any systemic manner.

The amino acid and manganese can be administered to the animal, preferably one in need of such administration, in any one of many ways, such as oral, parenteral, and the like, although oral is preferred. The amino acid and manganese can be administered in a wet or dry diet, either incorporated therein or on the surface of any diet component, such as, by spraying or precipitation thereon. They can be present in the nutritional diet per se or in a snack, supplement or a treat. They can also be present in the liquid portion of the diet such as water or another fluid. They can be administered as a powder, solid or as a liquid including a gel. If desired they can be orally administered in a pharmaceutical dosage form such as a capsule, tablet, caplet, syringe, and the like. Within the dosage form they can be present as a powder or a liquid such as a gel. Any of the usual pharmaceutical carriers can be employed such as water, glucose, sucrose and the like together with the amino acid and manganese. Although exemplified together, the amino acid and manganese can be administered separately, that is one in a diet and one in a liquid or a unit dose form, for example. Generally, they should be administered at least concomitantly, and preferably in the same carrier. When administered in a food, the sulfur containing amino acid and manganese can be administered as a compound, within the normal food constituents, or a combination of the two.

With respect to prevention of joint damage from arthritis, particularly osteo, or other noted conditions, a particular target group of pets, especially canines and felines, are those that would be in need of such preventative care as opposed to the general population. For example, pets, particularly large breed canines such as labrador retriever, rottweiler, german shepherd and the like are more susceptible to arthritis as demonstrated by its greater occurence in these pets. Additionally, pets above the age of six (6) years, particularly dogs and cats, have a significantly greater occurrence of arthritis, particularly osteo arthritis. Other examples of pets susceptible to the development of arthritis include horses. The invention can be additionally useful in treating animals especially canines and felines with arthritis, particularly osteo. Although exemplified with arthritis, other cartilage and joint affecting conditions, previously mentioned, are also applicable.

Various sulfur containing amino acids and their derivatives are applicable in the invention. These include D-methionine, L-methionine, DL-methionine, D-cysteine, L-cysteine, DL-cysteine, D-cystine, L-cystine, DL-cystine, S-adenosylmethionine, betaine, beta-hydroxy analog of methionine, and the like. The sulfur containing amino acid can be provided per se to the animal or can be present naturally in dietary materials such as fish meal, corn gluten meal, poultry meal, casein, manganese methionine (a chelate) and the like.

The manganese can be supplied to the animal in various forms including manganous sulfate, manganous oxide, manganous dioxide, manganous carbonate, manganous chloride, manganese proteinate, manganese chelate, manganese monoxide, manganese methionine, and the like.

The quantity of amino acid and manganese which should be employed for bringing about the effect(s) of the invention can vary substantially. All wt % are calculated on a dry matter basis of a daily diet sufficient to satisfy the nutrition needs of the animal. A minimum amount of the amino acid is above about 1.2 wt %, preferably above about 1.5 wt % and more preferably above about 1.8 wt %. The minimum amount of manganese is above about 50 ppm, preferably above about 75 ppm and more preferably above about 100 ppm. For example, a specific amount can be employed in the usual nutrient food ration on a daily basis or the same daily quantity can be provided to the animal in a treat or supplement on a daily basis. Additionally, a combination of these methods or any other dosing means can be employed as long as the effective quantity of sulfur containing amino acid and manganese is provided. Maximum quantities are any amount effective to reduce the quantity of cartilage abnormalities with little (acceptable level) or no toxicity. Examples of such quantities for the amino acid include not more than about 2.6 wt %, 2.3 wt % and 2.0 wt % on the same basis as for the minimums. Examples of such quantities of manganese include not more than about 200 ppm, preferably about 175 ppm and more preferably about 150 ppm on the same basis as the minimums.

As aforementioned, the amino acid and manganese can be in any food provided to the pet. Examples of such foods are regular diets providing all of the animal's nutrients, treats, supplements and the like. The actives can be provided in liquids or in pharmaceutical dosage forms such as capsules, tablets, pills, liquids or even parenterally administered through syringe. The most important aspect is that the pet be provided an effective amount of actives to reduce the abnormalities. The preferred route of aministration is oral and incorporated with a food. Foods are generally classified in the pet food industry as “wet” or “dry”. A wet food has a relatively high amount of water and is usually present in a can or a container wherein air is substantially or totally excluded. Examples of such foods are “chunk and gravy”, individual solid particles in the presence of a liquid gravy or a loaf type material which generally takes the shape of the receptacle. The dry food is generally a baked or preferably extruded material, the latter then cut into individual shaped portions, usually known as kibbles. The actives are readily incorporated into a wet food through conventional means.

With respect to pet food such as dog and cat the ranges of protein, fat and carbohydrate for a dog is 15-55 wt %, 5-40 wt %, 10-50 wt % respectively and for a cat is 15-55 wt %, 5-40 wt % and 10-50 wt % respectively.

Below are examples. These examples are illustrative exemplification of the broad scope of the invention.

Growing pigs (80 experimental units) were used as test model to determine the effect of methionine and manganese on cartilage abnormalities. The pigs were initially about 35 kg. Each pig was individually housed in 5.2 ft² pens with ad libitum access to food and water. The pigs were fed test foods for a period of 60 days to an approximate final weight of about 130 kg.

At the point of meat fabrication, the distal aspect of the right femur bone was collected and evaluated for gross- and histopathology.

The distal aspect of the right femor bone was preserved in formaldehyde, and stored at room temperature for gross observation. The joints were evaluated for the total number of lesions present on the joint surface (including clinical lesions, cartilage erosions, and abnormal growth patterns). Gross lesions were confirmed by histopathology characterization. Tissues sections were taken from the ventral weight barring aspects of the medial femoral condyle. Measures were evaluated on 2× and 10× photomicrographs to determine cell counts and to confirm pathological damage of the cartilage into the subchondral bone.

### EXAMPLES 1, 2 and 3

TABLE 1
Composition of Experimental Foods

| | Control | Example 1 | Example 2 | Example 3 |
|---|---|---|---|---|
| Corn | 71.00 | 78.50 | 71.00 | 71.00 |
| Soybean meal | 18.70 | 3.35 | 18.70 | 18.70 |
| Corn Starch | 3.78 | 3.00 | 2.52 | 2.48 |
| Ch White Grease | 3.00 | 1.00 | 3.00 | 3.00 |
| Dical | 1.97 | 1.13 | 1.98 | 2.03 |
| Limestone | 0.62 | 0.28 | 0.77 | 0.74 |
| Salt | 0.43 | 0.31 | 0.55 | 0.55 |
| L-lysine | 0.15 | 0.08 | 0.15 | 0.15 |
| Vitamin premix | 0.10 | 0.10 | 0.10 | 0.10 |
| Choline | 0.10 | 0.10 | 0.10 | 0.10 |
| TM premix | 0.10 | 0.10 | 0.10 | 0.10 |
| Mn sulfate | | 0.02 | | 0.02 |
| Tryptophan | | 0.03 | | |
| Poultry Meal | | 12.00 | | |
| DL-methionine | 0.04 | | 1.03 | 1.03 |
| Total | 100 | 100 | 100 | 100 |
| 100% DM basis | | | | |
| ME. Kcal/kg | 3604 | 3634 | 3604 | 3604 |
| Ca, % | 0.86 | 0.85 | 0.86 | 0.86 |
| P, % | 0.74 | 0.74 | 0.74 | 0.74 |
| Na, % | 0.22 | 0.22 | 0.22 | 0.22 |
| Lys, % | 0.97 | 0.96 | 0.97 | 0.97 |
| TSAA, % | 0.58 | 0.60 | 1.71 | 1.71 |
| Trp, % | 0.20 | 0.20 | 0.20 | 0.20 |
| Thr, % | 0.66 | 0.70 | 0.66 | 0.66 |
| Iso, % | 0.65 | 0.65 | 0.65 | 0.65 |
| Sulfur, ppm | 1664 | 2229 | 4147 | 4238 |
| Manganese, ppm | 41.3 | 107.8 | 41.2 | 127.4 |

TABLE 2
Analytical analyses of experimental foods - lot 1

| | Control | Example 1 | Example 2 | Example 3 |
|---|---|---|---|---|
| Crude protein | 17.32 | 18.34 | 16.63 | 16.93 |
| Fat | 7.76 | 7.58 | 7.46 | 7.42 |
| Fiber | 2.05 | 1.73 | 2.23 | 2.37 |
| Methionine + Cystine | 0.70 | 0.72 | 1.51 | 1.78 |
| Manganese | 46.4 | 81.2 | 43.4 | 110.0 |

TABLE 3
Analytical analyses of experimental foods - lot 2

| | Control | Example 1 | Example 2 | Example 3 |
|---|---|---|---|---|
| Crude protein | 17.38 | 18.43 | 19.30 | 17.94 |
| Fat | 6.83 | 7.89 | 7.54 | 7.46 |
| Fiber | 2.91 | 1.82 | 2.47 | 2.22 |
| Methionine + Cystine | 0.68 | 0.78 | 1.61 | 1.56 |
| Manganese | 41.8 | 96.8 | 42.2 | 110.1 |

TABLE 4
Effect of nutrients on cartilage abnormalities

| | Control | Example 1 | Example 2 | Example 3 |
|---|---|---|---|---|
| Total lesions | 2.38 | 2.25 | 1.38 | 0.88 |

As shown by the data, a combination of the sulfur containing amino acid and manganese (manganous ion) are required to statistically reduce the number of visually observed abnormalities (lesions and erosions) abnormalities of the cartilage, see Example 3. Neither examples 1 nor 2 bring about a statistically significant reduction in abnormalities. Example 1 is high in manganese but approximately the same in sulfur containing amino acid as control. Example 2 is high in sulfur containing amino acid but approximately the same in manganese as the control.

1. A method for decreasing cartilage abnormalities in an animal in need of such decrease which comprises systemically administering to said animal a cartilage abnormality decreasing effective amount of a combination of at least one sulfur containing amino acid and manganese.

2. A method in accordance with claim 1 wherein the animal is prevented from having or has a condition selected from the group consisting of osteoarthritis, rheumatoid arthritis, osteochondrosis, degenrative joint disease, synovitis, bacterial purulent arthritis, osteoarthropathia, and psoriatica.

3. A method in accordance with claim 1 wherein at least one sulfur containing amino acid is selected from the group consisting of D-methionine, L-methionine, DL-methionine, D-cysteine, L-cysteine, DL-cysteine, D-cystine, L-cystine, DL-cystine, S-adenosylmethionine, betaine, beta-hydroxy analog of methionine, and magnanese methionine.

4. A method in accordance with claim 1 wherein the minimum quantity of amino acid is about 1.2 wt % and the minimum amount of manganese is about 50 ppm.

5. A method in accordance with claim 1 wherein the administration is oral.

6. A composition suitable for systemic administration to a animal comprising a cartilage abnormality decreasing amount of a combination of at least one sulfur containing amino acid and manganese in association with a carrier.

7. The composition in accordance with claim 6 wherein at least one sulfur containing amino acid is selected from the group consisting of D-methionine, L-methionine, DL-methionine, D-cysteine, L-cysteine, DL-cysteine, D-cystine, L-cystine, DL-cystine, S-adenosylmethionine, betaine, beta-hydroxy analog of methionine, and magnanese methionine.

8. The composition in accordance with claim 6 wherein the administration is oral.

9. The composition in accordance with claim 6 wherein the minimum quantity of amino acid is about 1.2 wt % and the minimum amount of manganese is about 50 ppm.

10. A method for preventing degradation of cartilage tissue in an animal in need of said prevention which comprises administering to the said animal, a cartilage degradation prevention effective amount of at least one sulfur containing amino acid and manganese.

11. The method in accordance with claim 10 wherein the animal is prevented from having or has a condition selected from the group consisting of osteoarthritis, rheumatoid arthritis, osteochondrosis, degenrative joint disease, synovitis, bacterial purulent arthritis, osteoarthropathia, and psoriatica.

12. The method in accordance with claim 10 wherein at least one sulfur containing amino acid is selected from the group consisting of D-methionine, L-methionine, DL-methionine, D-cysteine, L-cysteine, DL-cysteine, D-cystine, L-cystine, DL-cystine, S-adenosylmethionine, betaine, beta-hydroxy analog of methionine, and magnanese methionine.

13. The method in accordance with claim 10 wherein the minimum quantity of amino acid is about 1.2 wt % and the minimum amount of manganese is about 50 ppm.

14. The method in accordance with claim 10 wherein the administration is oral.

15. A method for enhancing cartilage development in an animal which comprises administering to the said animal an enhancing cartilage development effective amount of at least one sulfur containing amino acid and manganese.

16. A method for preventing disease associated with cartilage degradation in an animal which comprises administering to the said animal an enhancing cartilage development effective amount of at least one sulfur containing amino acid and manganese.

17. A method for treating disease associated with cartilage degradation in an animal which comprises administering to the said animal an enhancing cartilage development effective amount of at least one sulfur containing amino acid and manganese.
